(19) United States
(12) Patent Application Publication
(10) Pub. No.: US 2016/0022845 A1
(43) Pub. Date: Jan. 28, 2016

(54) SYNTHON COMPOSITION

(71) Applicant: GE HEALTHCARE LIMITED, BUCKINGHAMSHIRE (GB)

(72) Inventors: RAJIV BHALLA, ST. LUCIA (AU); ALEXANDER JACKSON, AMERSHAM (GB); GARETH SMITH, AMERSHAM (GB)

(73) Assignee: GE HEALTHCARE LIMITED, BUCKINGHAMSHIRE (GB)

(21) Appl. No.: 14/387,660

(22) PCT Filed: Mar. 28, 2013
(86) PCT No.: PCT/EP2013/056720

§ 371 Date: Sep. 24, 2014

Related U.S. Application Data
(60) Provisional application No. 61/617,698, filed on Mar. 30, 2012.

(30) Foreign Application Priority Data
Mar. 30, 2012 (GB) 1205703.0

(51) Int. Cl. A61K 51/04; A61K 51/12

(52) U.S. Cl. CPC A61K 51/0497; A61K 51/1282

(57) ABSTRACT

The present invention relates to an improved [¹⁸F]labelled synthon composition, wherein the non-radioactive impurities in said composition have been found to be more straightforward to remove than with known compositions comprising said [¹⁸F]labelled synthon. The resultant purified [¹⁸F]label led synthon therefore can be used in the production of a positron emission tomography (PET) tracer having improved properties for in vivo imaging. The invention also includes methods of imaging and/or diagnosis using the radiopharmaceutical compositions described.

### TECHNICAL FIELD OF THE INVENTION

The present invention relates to an improved [¹⁸F]labelled synthon composition, wherein the non-radioactive impurities in said composition have been found to be more straightforward to remove than with known compositions comprising said [¹⁸F]labelled synthon. The resultant purified [¹⁸F]labelled synthon therefore can be used in the production of a positron emission tomography (PET) tracer having improved properties for in vivo imaging. The invention also includes methods of imaging and/or diagnosis using the radiopharmaceutical compositions described.

### DESCRIPTION OF RELATED ART

The widespread availability of [¹⁸F]fluoride, its optimal half-life (110 min) and low positron energy (0.64 MeV), makes it the isotope of choice for positron emission tomography (PET) imaging (Snyder & Kilbourn 2003 “Handbook of Radiopharmaceuticals, Radiochemistry and Applications”, Welch & Redvanly, Eds; Chapter 6: 195-227).

Radiofluorination can be conveniently carried out via direct radiofluorination by reacting radiofluorine with a suitable precursor compound. A suitable precursor compound for direct radiofluorination may comprise a group selected for example from NO₂, trimethylammonium (NMe₃), Cl, Br, I, tosylate (OTs), mesylate (OMs), nosylate (ONs) and triflate (OTf). Carroll et al (2005 J Label Comp Radiopharm; 48(7): 519-520 and 2007 J Fluorine Chem; 128(2): 127-132). Lehmann et al (WO2010066380) describe the synthesis of compounds labelled with ¹⁸F by direct labelling of a sulfonium-derivatised precursor compound.

Although simple to perform, direct radioiodination has disadvantages, especially when applied to the radioiodination of biomolecules such as proteins. More preferred in this case are generic radiolabeling strategies using prosthetic groups (also known as “synthons”). These offer the advantage that a significant part of the radiochemical process can be standardized and applied to multiple products. The prosthetic group must be unreactive toward any functional groups found in the product in order to form a stable bond in a site-specific manner. The aforementioned criteria are met by utilizing oxime bond formation between an [¹⁸F]aldehyde and an aminooxy-modified peptide. This type of chemoselective ligation chemistry has been widely employed using 4-[¹⁸F]fluorobenzaldehyde as the prosthetic group with acceptable yields being reported for a range of [¹⁸F]labelled peptides (Cuthbertson et al WO2004080492; Poethko et al 2004 J Nuc Med; 45: 892-902; Lee et al 2005 J Label Comp Radiopharm; 48: S288).

Glaser et al (2008 Bioconj Chem; 19(4): 951-957) describe the synthesis of [¹⁸F]labelled aldehydes, including [¹⁸F]fluorobenzaldehyde ([¹⁸F]FBA), and their conjugation to amino-oxy functionalised cyclic RGD peptides. Glaser et al describe that [¹⁸F]FBA is obtained by radiofluorination of 4-N,N,N-trimethylammonium benzaldehyde trifluoromethanesulfonate as illustrated in the following reaction:

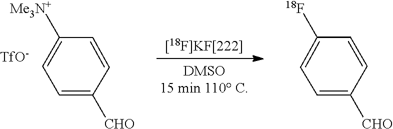

Battle et al (2011 J Nucl Med; 52(3): 424-430) disclose purification of [¹⁸F]FBA by diluting with water, and trapping on a solid-phase extraction (SPE) cartridge. Impurities such as precursor, DMSO, Kryptofix-222 and hydrophilic by-products were said to be eluted to waste, and the [¹⁸F]FBA subsequently eluted with ethanol.

The present inventors have, however, found that using the SPE method of Battle et al only some of the precursor is eluted to waste, and the remainder co-elutes when the [¹⁸F]FBA is eluted with ethanol. There is therefore still a need for alternative methods of labelling biological targeting moieties with ¹⁸F.

### SUMMARY OF THE INVENTION

The present invention relates to a composition comprising an [¹⁸F]labelled synthon wherein impurities which affect imaging in vivo and found in known compositions of said synthon are not present. Also provided is a radiopharmaceutical composition obtained by means of said synthon. The invention also includes methods of imaging and/or diagnosis using the radiopharmaceutical compositions described.

### DETAILED DESCRIPTION OF THE INVENTION

In a first aspect, the present invention provides a composition comprising:
an [¹⁸F]labelled synthon of Formula X:

¹⁸F—Ar^{x}—X¹ (X)
wherein X¹ is CR¹O wherein R¹ is hydrogen or C₁₋₆ alkyl; and, Ar^{x} is 6-membered aromatic ring comprising between 0-3 nitrogen heteroatoms;
together with one or more non-radioactive compounds selected from:
   (i) compounds of Formula Y:

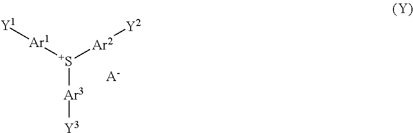

wherein Ar¹ is the same as A^{x} and Ar² and Ar³ are the same and are as defined for A^{x}; A⁻ is a corresponding counter anion to the sulfonium cation; and, Y¹ is the same as X¹ and Y² and Y³ are the same and are either hydrogen or —CR¹O as defined for Formula X; and,
(ii) compounds of Formula Z:

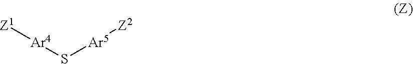

wherein each of Ar⁵ and Ar⁶ is a 6-membered aromatic ring comprising between 0-3 nitrogen heteroatoms; and, each of Z¹ and Z² is hydrogen or —CR¹O as defined for Formula X.

The term “a composition comprising” refers to a chemical composition having the components listed, but that other, unspecified compounds or species may be present in addition. A preferred subset can therefore be “a composition consisting essentially of”, which means that the composition has the components listed without other compounds or species being present.

An “[¹⁸F]labelled synthon”, also known as an [¹⁸F]labelled prosthetic group, is a small molecule labelled with ¹⁸F that may be coupled with a non-radioactive precursor compound to result in the desired [¹⁸F]labelled product.

The term “alkyl” used either alone or as part of another group is defined as any straight, branched or cyclic, saturated or unsaturated CₙH₂ₙ₊₁ group.

The term “6-membered aromatic ring” refers to an aromatic substituent based on benzene (C₆H₆) comprising 0-3 nitrogen heteroatoms. A “nitrogen heteroatom” is a nitrogen that takes the place of a CH in the aromatic ring. Examples of 6-membered aromatic rings of the invention include phenyl, pyridyl, and pyrimidyl.

The term “non-radioactive compounds” refers to any compound that comprises no radioactive atoms.

The term “counter anion” refers to an anion that accompanies a cationic species in order to maintain electric neutrality. An “anion” is an ion with more electrons than protons, giving it a net negative charge. Any anion may be used as the counter anions. Non-limiting examples include CF₃SO₃⁻, PF₆⁻, BF₄⁻, and AsF₆⁻, SO₄²⁻, and NO₃⁻.

X¹ is preferably —CR¹O wherein R¹ is hydrogen or C₁₋₃ alkyl, and is most preferably —CHO.

Ar¹ is preferably phenyl or pyridyl, most preferably phenyl.

Ar² is preferably phenyl or pyridyl, most preferably phenyl.

Ar³ and Ar⁴ are preferably both phenyl or both pyridyl, most preferably both phenyl.

Y¹ is preferably —CR¹O wherein R¹ is hydrogen or C₁₋₃ alkyl, and is most preferably —CHO.

Y² and Y³ are both preferably hydrogen.

Y² and Y³ are alternatively preferably —CR¹O wherein R¹ is hydrogen or C₁₋₆ alkyl, and is most preferably —CHO.

A⁻ is preferably selected from CF₃SO₃⁻, PF₆⁻, BF₄⁻, and AsF₆⁻.

Ar⁵ and Ar⁶ are preferably either phenyl or pyridyl, most preferably phenyl.

Z¹ and Z² are preferably hydrogen or —CHO.

For a preferred compound of Formula X:

X¹ is CR¹O wherein R¹ is hydrogen; and,

Ar¹ is phenyl or pyridyl and is most preferably phenyl.

For a most preferred compound of Formula X:

X¹ is CR¹O wherein R¹ is hydrogen; and,
Ar¹ is phenyl.

For a preferred compound of Formula Y:

Ar² is phenyl or pyridyl;

Ar³ and Ar⁴ are the same and are either both phenyl or both pyridyl;

Y¹ is CR¹O wherein R¹ is hydrogen;

Y² and Y³ are the same and are either both hydrogen or both CR¹O wherein R¹ is hydrogen; and,

A⁻ is selected from CF₃SO₃⁻, PF₆⁻, BF₄⁻, and AsF₆⁻.

For a most preferred compound of Formula Y:

Ar² is phenyl;

Ar³ and Ar⁴ are both phenyl;

Y¹ is CR¹O wherein R¹ is hydrogen;

Y² and Y³ are both hydrogen; and,

A⁻ is selected from CF₃SO₃⁻, PF₆⁻, BF₄⁻, and AsF₆⁻.

For an alternative most preferred compound of Formula Y:

Ar² is phenyl;

A³ and Ar⁴ are both phenyl;

Y¹ is CR¹O wherein R¹ is hydrogen;

Y² and Y³ are both CR¹O wherein R¹ is hydrogen; and,

A⁻ is selected from CF₃SO₃⁻, PF₆⁻, BF₄⁻, and AsF₆⁻.

For a preferred compound of Formula Z:

Ar⁵ and Ar⁶ are independently either phenyl or pyridyl;

Z¹ and Z² are independently hydrogen or —CHO

Preferably for the composition of the invention defined hereinabove:
said compound of Formula X is a compound of Formula Xa:

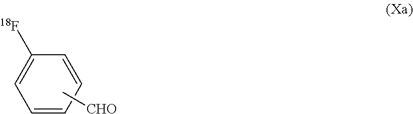

said compound of Formula Y is a compound of Formula Ya:

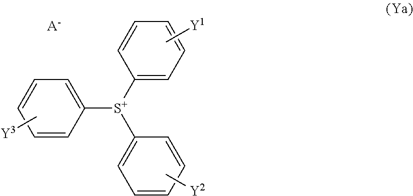

wherein Y¹⁻³ are as defined for Formula Y; and,
said compound of Formula Z is a compound of Formula Za:

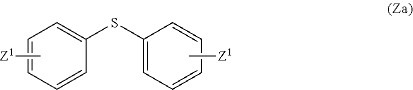

wherein Z¹ and Z² are as defined for Formula Z.

For the above-defined composition it is preferred that X¹ and Y¹ are both located at the ortho-position. In an alternative preferred embodiment, it is preferred that X¹ and Y¹ are both located at the para-position.

The composition of the present invention is advantageous over known compositions that comprise a compound of Formula X. One well-known compound of Formula X is [¹⁸F]fluorobenzaldehyde ([¹⁸F]FBA), which is frequently used for the radiofluorination of peptides. In the known process described by Battle et al (2011 J Nucl Med; 52(3): 424-430) a major chemical impurity is formed:

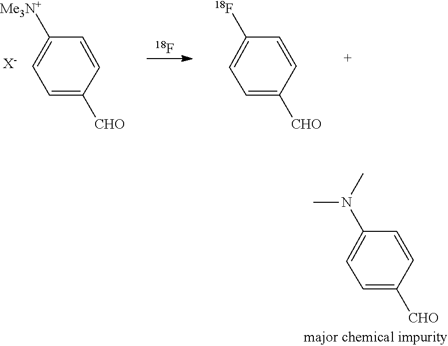

The present inventors have found that this major chemical impurity is not completely removed following solid-phase extraction (SPE).

In contrast, in the composition of the present invention, the compounds of Formula Y and Formula Z are very straightforward to remove from the above-described composition of the present invention to provide pure compound of Formula X. In turn, a compound of Formula X which does not include the major chemical impurity shown above can be used to obtain a radiofluorinated product having an improved purity profile.

The above-described composition of the present invention is obtained by the reaction of a compound of Formula Y with [¹⁸F]fluoride. Accordingly, in a second aspect of the present invention is provided a method to prepare the composition as defined for the first aspect of the invention wherein said method comprises:
(i) reaction of a non-radioactive compound of Formula Y as defined above with [¹⁸F]fluoride; and,
(ii) purification to result in said composition.

Certain compounds of Formula Y may be obtained by use of methods known in the art. Crivello & Lam (1978 J Org Chem; 43(15): 3055-3058), Crivello (U.S. Pat. No. 4,161,478) and Yanez et al (2009 Chem Comm: 827-829) each provide teachings as to how to obtain a variety of compounds of Formula Y by reaction of a compound of Formula Z with a diaryliodonium salt of Formula Q as follows:

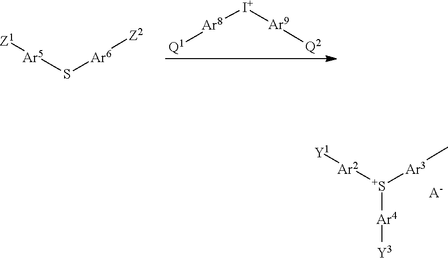

wherein the features of Formula Y and Formula Z are as defined herein, Q¹ and Q² are as defined respectively herein for Z¹ and Z², and Ar⁸ and Ar⁹ are as defined respectively herein for Ar⁵ and Ar⁶. The prior art methods can be adapted in a straightforward manner using routine skill in the art to obtain any compound falling within the definition of Formula Y.

[¹⁸F]Fluoride used in the method of the second aspect of the invention is normally obtained as an aqueous solution from the nuclear reaction ¹⁸O(p,n)¹⁸F. Once it is made reactive by drying and by the addition of a cationic counterion and the removal of water ¹⁸F⁻ can be reacted with said compound of Formula Y. The step of “drying” [¹⁸F]fluoride comprises evaporation of water to result in anhydrous [¹⁸F]fluoride. This drying step are suitably carried out by application of heat and use of a solvent such as acetonitrile to provide a lower boiling azeotrope. A “cationic counterion” is a positively-charged counterion examples of which include large but soft metal ions such as rubidium or caesium, potassium complexed with a cryptand, or tetraalkylammonium salts. A preferred cationic counterion is a metal complex of a cryptand, most preferably wherein said metal is potassium and wherein said cryptand is Kryptofix 222.

The term “purification” refers to separation of the [¹⁸F]labelled synthon of Formula X from the non-radioactive compounds of Formula Y and Formula Z comprised in the composition of the first aspect of the invention with the aim of obtaining pure [¹⁸F]labelled synthon of Formula X. The purification step of the method of the invention is suitably carried out by chromatography or solid-phase extraction (SPE), wherein said chromatography is preferably high-performance liquid chromatography (HPLC). Purification is facilitated by virtue of the fact that the non-radioactive compounds of Formula Y are charged and as such easy to remove by ion exchange, and also that the non-radioactive compounds of Formula Z are more lipophilic than the [¹⁸F]labelled synthon of Formula X and as such they can be removed using differential lipophilicity to purify using solid-phase extraction (SPE). Purification is even more straightforward where a symmetrical compound of Formula Y is used in the method as even fewer non-radioactive compounds are generated in the resultant composition.

The method of second aspect of the invention is preferably carried out on an automated synthesis apparatus. By the term “automated synthesis apparatus” is meant an automated module based on the principle of unit operations as described by Satyamurthy et al (1999 Clin Positr Imag; 2(5): 233-253). The term ‘unit operations” means that complex processes are reduced to a series of simple operations or reactions, which can be applied to a range of materials. Such automated synthesis apparatuses are preferred for the method of the present invention especially when a radiopharmaceutical composition is desired. They are commercially available from a range of suppliers (Satyamurthy et al, above), including: GE Healthcare; CTI Inc; Ion Beam Applications S.A. (Chemin du Cyclotron 3, B-1348 Louvain-La-Neuve, Belgium); Raytest (Germany) and Bioscan (USA).

A commercial automated synthesis apparatus also provides suitable containers for the liquid radioactive waste generated as a result of the radiopharmaceutical preparation. Automated synthesis apparatuses are not typically provided with radiation shielding, since they are designed to be employed in a suitably configured radioactive work cell. The radioactive work cell provides suitable radiation shielding to protect the operator from potential radiation dose, as well as ventilation to remove chemical and/or radioactive vapours. The automated synthesis apparatus preferably comprises a cassette. By the term “cassette” is meant a piece of apparatus designed to fit removably and interchangeably onto an automated synthesis apparatus, in such a way that mechanical movement of moving parts of the synthesizer controls the operation of the cassette from outside the cassette, i.e. externally. Suitable cassettes comprise a linear array of valves, each linked to a port where reagents or vials can be attached, by either needle puncture of an inverted septum-sealed vial, or by gas-tight, marrying joints. Each valve has a male-female joint which interfaces with a corresponding moving arm of the automated synthesis apparatus. External rotation of the arm thus controls the opening or closing of the valve when the cassette is attached to the automated synthesis apparatus. Additional moving parts of the automated synthesis apparatus are designed to clip onto syringe plunger tips, and thus raise or depress syringe barrels.

The cassette is versatile, typically having several positions where reagents can be attached, and several suitable for attachment of syringe vials of reagents or chromatography cartridges (e.g. for SPE). The cassette always comprises a reaction vessel. Such reaction vessels are preferably 0.5 to 10 mL, more preferably 0.5 to 5 mL and most preferably 0.5 to 4 mL in volume and are configured such that 3 or more ports of the cassette are connected thereto, to permit transfer of reagents or solvents from various ports on the cassette. Preferably the cassette has 15 to 40 valves in a linear array, most preferably 20 to 30, with being especially preferred. The valves of the cassette are preferably each identical, and most preferably are 3-way valves. The cassettes are designed to be suitable for radiopharmaceutical manufacture and are therefore manufactured from materials which are of pharmaceutical grade and ideally also are resistant to radiolysis.

Preferred automated synthesis apparatuses of the present invention comprise a disposable or single use cassette which comprises all the reagents, reaction vessels and apparatus necessary to carry out the preparation of a given batch of radiofluorinated radiopharmaceutical. The cassette means that the automated synthesis apparatus has the flexibility to be capable of making a variety of different radiopharmaceuticals with minimal risk of cross-contamination, by simply changing the cassette. The cassette approach also has the advantages of: simplified set-up hence reduced risk of operator error; improved GMP (Good Manufacturing Practice) compliance; multi-tracer capability; rapid change between production runs; pre-run automated diagnostic checking of the cassette and reagents; automated barcode cross-check of chemical reagents vs the synthesis to be carried out; reagent traceability; single-use and hence no risk of cross-contamination, tamper and abuse resistance.

In a third aspect, the present invention provides a method to prepare a composition comprising a positron emission tomography (PET) tracer of Formula V:

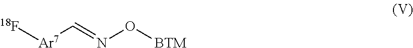

wherein Ar⁷ is as defined in above for the first aspect of the invention for Ar¹ and BTM is a biological targeting molecule;
wherein said method comprises the method as defined above for the second aspect of the invention as well as the additional subsequent step of reacting said [¹⁸F]labelled synthon of Formula X with a precursor compound of Formula W:

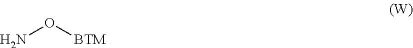

wherein BTM is as defined for Formula V.

Similarly to the method of the second aspect of the invention, the method of the third aspect of the invention is preferably carried out on an automated synthesis apparatus.

By the term “biological targeting molecule” (BTM) is meant a compound which, after administration, is taken up selectively or localises at a particular site of the mammalian body in vivo. Such sites may be implicated in a particular disease state or be indicative of how an organ or metabolic process is functioning. The BTM may be of synthetic or natural origin, but is preferably synthetic. The term “synthetic” has its conventional meaning, i.e. man-made as opposed to being isolated from natural sources e.g. from the mammalian body. Such compounds have the advantage that their manufacture and impurity profile can be fully controlled. The molecular weight of the BTM is preferably up to 10000 Daltons. More preferably, the molecular weight is in the range 200 to 9000 Daltons, most preferably 300 to 8000 Daltons, with 400 to 6000 Daltons being especially preferred. When the BTM is a non-peptide, the molecular weight of the BTM is preferably up to 3000 Daltons, more preferably 200 to 2500 Daltons, most preferably 300 to 2000 Daltons, with 400 to 1500 Daltons being especially preferred. By the term “peptide” is meant a compound comprising two or more amino acids, as defined below, linked by a peptide bond (i.e. an amide bond linking the amine of one amino acid to the carboxyl of another). When the BTM is an enzyme substrate, enzyme antagonist, enzyme agonist, enzyme inhibitor or receptor-binding compound it is preferably a non-peptide, and more preferably is synthetic. By the term “non-peptide” is meant a compound which does not comprise any peptide bonds, i.e. an amide bond between two amino acid residues.

The method of the third aspect is preferably carried out in a sterile manner, such that a pharmaceutical composition comprising said PET tracer of Formula V is obtained. The radiopharmaceutical compositions of the present invention may be prepared by various methods:
(i) aseptic manufacture techniques in which the ¹⁸F-radiolabelling step is carried out in a clean room environment;
(ii) terminal sterilisation, in which the ¹⁸F-radiolabelling is carried out without using aseptic manufacture and then sterilised at the last step [e.g. by gamma irradiation, autoclaving dry heat or chemical treatment (e.g. with ethylene oxide)];
(iii) kit methodology in which a sterile, non-radioactive kit formulation comprising a suitable precursor and optional excipients is reacted with a suitable supply of ¹⁸F;
(iv) aseptic manufacture techniques in which the ¹⁸F-radiolabelling step is carried out using an automated synthesizer apparatus.

Method (iv) is preferred.

The term “pharmaceutical composition” refers to a composition comprising said PET tracer of Formula V together with a biocompatible carrier in a form suitable for mammalian administration.

By the phrase “in a form suitable for mammalian administration” is meant a composition which is sterile, pyrogen-free, lacks compounds which produce toxic or adverse effects, and is formulated at a biocompatible pH (approximately pH 4.0 to 10.5). Such compositions lack particulates which could risk causing emboli in vivo, and are formulated so that precipitation does not occur on contact with biological fluids (e.g. blood). Such compositions also contain only biologically compatible excipients, and are preferably isotonic.

The “biocompatible carrier” is a fluid, especially a liquid, in which the PET tracer of Formula V can be suspended or preferably dissolved, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous buffer solution comprising a biocompatible buffering agent (e.g. phosphate buffer); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). Preferably the biocompatible carrier is pyrogen-free water for injection, isotonic saline or phosphate buffer.

In a fourth aspect the present invention provides a cassette for carrying out the method of the second aspect of the invention on an automated synthesis apparatus, said cassette comprising
(i) a vessel containing the compound of Formula Y as defined above for the first aspect of the invention; and
(ii) means for eluting the vessel with a suitable source of [¹⁸F]fluoride; and optionally,
(iii) an ion-exchange cartridge for removal of excess [¹⁸F]fluoride.

In a fifth aspect the present invention provides a cassette for carrying out the method of the third aspect of the invention on an automated synthesis apparatus, said cassette comprising the features of the cassette as defined for the fourth aspect of the invention in addition to (iv) a vessel containing said compound of Formula W as defined for the third aspect of the invention.

A sixth aspect of the present invention is a pharmaceutical composition as defined hereinabove comprising the PET tracer of Formula V as defined for the third aspect of the invention wherein said pharmaceutical composition is obtained according to the method of the third aspect of the invention.

In a seventh aspect, the present invention provides a method of imaging the human or animal body which comprises generating a PET image of at least a part of said body to which the pharmaceutical composition of the sixth aspect of the invention has distributed.

In a preferred embodiment, said method of imaging is carried out repeatedly to monitor the effect of treatment of a human or animal body with a drug, said imaging being effected before and after treatment with said drug, and optionally also during treatment with said drug.

Alternatively, said method of the seventh aspect of the invention can be understood as wherein said pharmaceutical composition has been previously administered to said body.

In an eighth aspect, the present invention provides a method of diagnosis of the human or animal body which comprises the imaging method of the seventh aspect of the invention.

Alternatively said eighth aspect can be understood to be the pharmaceutical composition of the sixth aspect of the invention for use in said method of diagnosis.

The invention is illustrated by the non-limiting Example detailed below.

### BRIEF DESCRIPTION OF THE EXAMPLES

Example 1 describes the synthesis of an asymmetrical sulfonium precursor compound of the present invention.

Example 2 describes ¹⁸F labelling of an asymmetric sulfonium precursor compound of the present invention.

### LIST OF ABBREVIATIONS USED IN THE EXAMPLES

[¹⁸F]FBA [¹⁸F]fluorobenzadehyde
HPLC high-performance liquid chromatography
min minute(s)
QMA quaternary methylammonium
RP reverse phase
SPE solid phase extraction
UV ultraviolet

### Example 1

### Preparation of (4-formylphenyl)diphenylsulfonium hexafluorophosphate

In a 5 mL glass reaction vessel 4-phenylthiobenzaldehyde (1 g, 4.67 mmol), diphenyliodonium hexafluoro phosphate (4 g, 9.39 mmol) and copper(II) benzoate (0.12 g) were mixed in the dark in chlorobenzene (2 mL) under N₂ atmosphere. The resulting mixture was heated to 125° C. for 15 min under microwave. Upon completion of reaction the solvent was evaporated under vacuum and isolated the crude product as a dark yellow residue. Purified the crude product using reversed phase chromatography: Zorbax SB-C18, 9.4× 50 mm, 5μ column, Gradient: Solvent A: Water, Solvent B: Acetonitrile; flow 10 ml/min, gradient: 98/2(A/B) isocratic for 2 min, 20/80 over 8 min, isocratic for 2 min, 98/2 in 2 min. Isolated the 98.8% pure material as a white solid (0.5 g).

¹H NMR (500 MHz, acetone-d6): 10.24 (1H, s), 8.34 (2H, d, 9 Hz), 8.15 (2H, d, J=9 Hz,), 8.08 (6H, m), 7.91 (4H, t, J=9 Hz)

m/z calculated for: 291.08; found, 291.4

### Example 2

### ¹⁸F labelling of (4-formylphenyl)diphenylsulfonium 2,2,2-trifluoroacetate

[¹⁸F]fluoride (370 MBq) was diluted with water (1 mL) and trapped a Waters QMA carb. Cartridge. The [¹⁸F]fluoride was eluted into a TRACERlab™ reaction vessel with a solution containing tetrabutylammonium carbonate in acetonitrile/water. The [¹⁸F]fluoride solution was dried under vacuum and with a stream of nitrogen. (4-formylphenyl)diphenylsulfonium 2,2,2-trifluoroacetate (8.5 mg) in dimethylsulfoxide (1 ml) was added to the resultant [¹⁸F]tetrabutylammonium fluoride residue and heated in the sealed reactor for 15 minutes at 130° C.

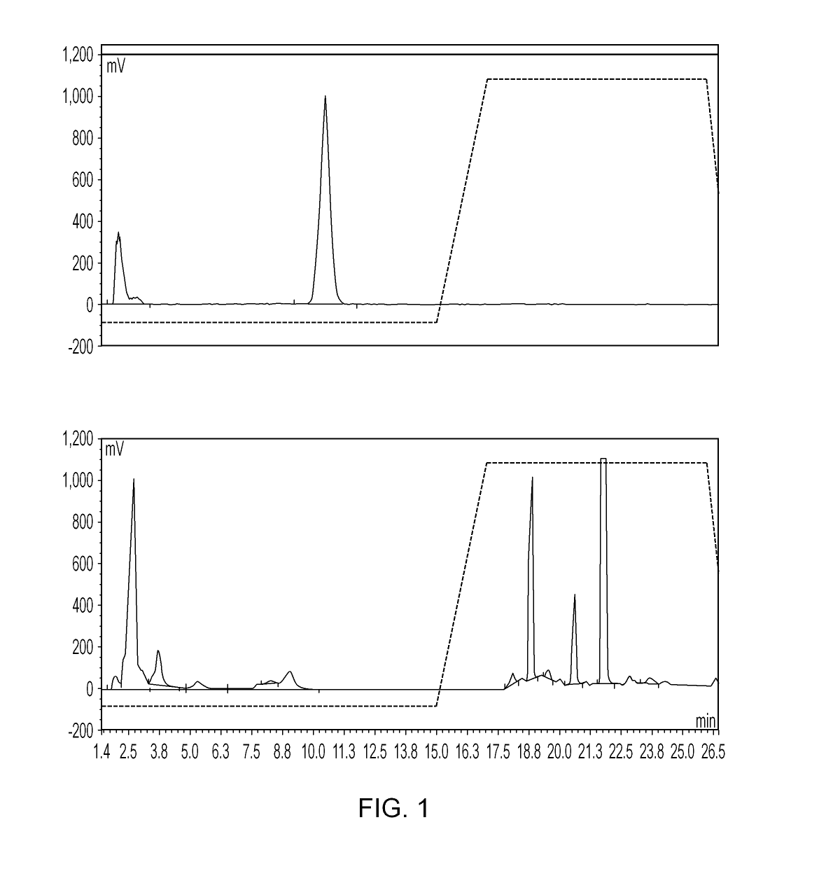

The contents of the reactor were then cooled to 50° C. and diluted with 70:30 water:dimethylsulfoxide. A sample of the clear-yellow crude product solution was submitted to analytical RP HPLC (A=water, B=acetonitrile, 30% B for 15 minutes to 95% B) and an incorporation of ˜78% was determined with [¹⁸F]fluorobenzaldehyde ([¹⁸F]-FBA) eluting in 10.478 minutes (see FIG. 1 wherein the radioactive trace is top and the UV trace is bottom). The early peaks in the UV are charged species from the reaction mixture and the species eluting during the 95% B wash are more lipophilic by-products, probably diphenylsulfane and 4-(phenylthio)benzaldehyde. The HPLC shows that [¹⁸F]FBA is separated from any UV impurities and those than run close are minor in comparison to the bulk chemical in the crude product.

What is claimed is:

1. A composition comprising:
an [¹⁸F]labelled synthon of Formula X:
¹⁸F—Ar¹—X¹ (X)
wherein X¹ is —CR¹O wherein R¹ is hydrogen or C₁₋₆ alkyl; and, Ar¹ is 6-membered aromatic ring comprising between 0-3 nitrogen heteroatoms;
together with one or more non-radioactive compounds selected from:
(i) compounds of Formula Y: 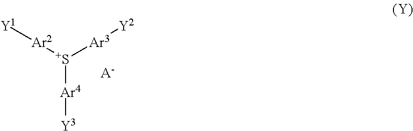
wherein Ar² is the same as Ar¹ and Ar³ and Ar⁴ are the same and are as defined for Ar¹; A⁻ is a corresponding counter anion to the sulfonium cation; and, Y¹ is the same as X¹ and Y² and Y³ are the same and are either hydrogen or —CR¹O as defined for Formula X; and,
(ii) compounds of Formula Z: 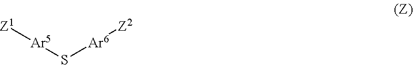
wherein each of Ar⁵ and Ar⁶ is a 6-membered aromatic ring comprising between 0-3 nitrogen heteroatoms; and, each of Z¹ and Z² is hydrogen or —CR¹O as defined for Formula X.

2. The composition as defined in claim 1 wherein R¹ is hydrogen.

3. The composition as defined in claim 1 wherein Ar¹ is phenyl or pyridyl.

4. The composition as defined in claim 3 wherein Ar¹ is phenyl.

5. The composition as defined in claim 1 wherein Ar² and Ar³ are both phenyl or pyridyl.

6. The composition as defined in claim 5 wherein Ar² and Ar³ are both phenyl.

7. The composition as defined in claim 1 wherein Y² and Y³ are both hydrogen.

8. The composition as defined in claim 1 wherein Y² and Y³ are both —CHO.

9. The composition as defined in claim 1 wherein said compound of Formula X is a compound of Formula Xa: 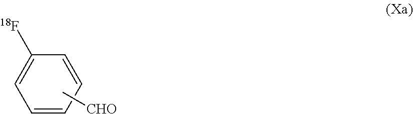
said compound of Formula Y is a compound of Formula Ya: 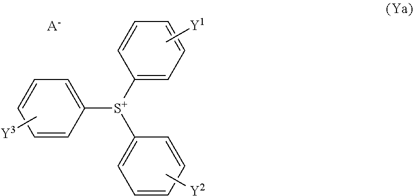
wherein Y¹⁻³ are as defined for Formula Y; and,
said compound of Formula Z is a compound of Formula Za: 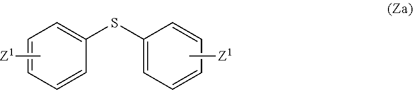
wherein Z¹ and Z² are as defined for Formula Z.

10. The composition as defined in claim 1 wherein X¹ and Y¹ are both located at the ortho-position.

11. The composition as defined in claim 1 wherein X¹ and Y¹ are both located at the para-position.

12. The composition as defined in claim 1 wherein A⁻ is selected from CF₃SO₃⁻, PF₆⁻, BF₄⁻, and AsF₆⁻.

13. A method wherein said method comprises:
(i) reaction of a non-radioactive compound of Formula Y as defined in claim 1 with [¹⁸F]fluoride; and,
(ii) purification to result in a composition as defined in claim 1.

14. The method as defined in claim 13 wherein said purification is carried out by high-performance liquid chromatography (HPLC).

15. The method as defined in claim 13 wherein said purification is carried out by solid-phase extraction (SPE).

16. The method as defined in claim 13 which is carried out on an automated synthesis apparatus.

17. A method as defined in claim 13 further comprising the step of reacting said [¹⁸F]labelled synthon of Formula X as defined in claim 1 with a precursor compound of Formula W: 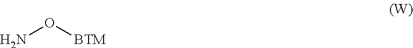
wherein BTM is a biological targeting molecule, to prepare a composition comprising a positron emission tomography (PET) tracer of Formula V: 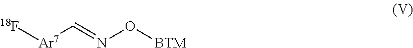
wherein Ar⁷ is as defined in claim 1 for Ar¹.

18. The method as defined in claim 17 which is carried out on an automated synthesis apparatus.

19. A cassette for carrying out the method as defined in claim 16, said cassette comprising
(i) a vessel containing the compound of Formula Y; 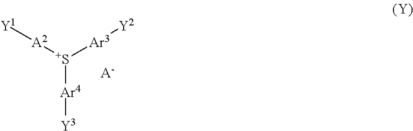
wherein Ar² is the same as Ar¹ and Ar³ and Ar⁴ are the same and are as defined for Ar¹; Ar¹ is 6-membered aromatic ring comprising between 0-3 nitrogen heteroatoms;
A⁻ is a corresponding counter anion to the sulfonium cation; and, Y¹ is the same as X¹ wherein X¹ is —CR¹O wherein R¹ is hydrogen or C₁₋₆alkyl; and Y² and Y³ are the same and are either hydrogen or —CR¹O wherein R¹ is as defined above; and
(ii) means for eluting the vessel with a suitable source of [¹⁸F]fluoride; and optionally,
(iii) an ion-exchange cartridge for removal of excess [¹⁸F]fluoride.

20. A cassette defined in claim 18, further comprising (iv) a vessel containing said compound of Formula W: 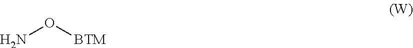
wherein BTM is a biological targeting molecule.

21. A pharmaceutical composition comprising the PET tracer of Formula V as defined in claim 17 wherein said pharmaceutical composition is obtained according to the method defined in claim 17.

22. A method of imaging the human or animal body which comprises generating a PET image of at least a part of said body to which the pharmaceutical composition of claim 21 has distributed.

23. The method of claim 22, which is carried out repeatedly to monitor the effect of treatment of a human or animal body with a drug, said imaging being effected before and after treatment with said drug, and optionally also during treatment with said drug.

24. The method of claim 22, wherein said pharmaceutical composition has been previously administered to said body.

25. A method of diagnosis of the human or animal body which comprises the imaging method of claim 22.

26. (canceled)
